(19) United States
(12) Patent Application Publication
(10) Pub. No.: US 2025/0229006 A1
(43) Pub. Date: Jul. 17, 2025

(54) 3D PRINTED ARTIFICIAL BILE DUCTS AND MANUFACTURING METHOD THEREOF

(71) Applicants: IUCF-HYU (INDUSTRY-UNIVERSITY COOPERATION FOUNDATION HANYANG UNIVERSITY), Seoul (KR); PASAN NATIONAL UNIVERSITY INDUSTRY-UNIVERSITY COOPERATION FOUNDATION, Busan (KR)

(72) Inventors: Dongho CHOI, Seoul (KR); Suk-Hee Park, Busan (KR); Ji Hyun Shin, Seoul (KR); Salas Silva Elsy Soraya, Seoul (KR); Yeong-Seo Kim, Bussan (KR)

(73) Assignee: IUCF-HYU (INDUSTRY-UNIVERSITY COOPEATION FOUNDATION HANYANG UNIVERSITY), Seoul (KR)

(21) Appl. No.: 19/020,111

(22) Filed: Jan. 14, 2025

(30) Foreign Application Priority Data
Jan. 17, 2024 (KR) 10-2024-0007417

(51) Int. Cl. A61L 27/54; A61F 2/00; A61F 2/04; A61L 27/26; A61L 27/56; A61L 27/58; B29C 33/38; B29C 33/52; B29C 64/118; B29L 31/00; B32B 5/02; B32B 5/20; B32B 5/24; B33Y 80/00

(52) U.S. Cl. CPC A61L 27/54; A61F 2/04; A61L 27/26; A61L 27/56; A61L 27/58; B29C 33/3842; B29C 64/118; B32B 5/022; B32B 5/20; B32B 5/245; B33Y 80/00; A61F 2002/0081; A61F 2002/041; A61F 2210/0004; A61F 2240/004; A61F 2250/0067; A61L 2300/222; A61L 2430/22; B29C 33/52; B29K 2829/04; B29L 2031/7532; B32B 2262/0276; B32B 2266/0278; B32B 2307/7163; B32B 2535/00

(57) ABSTRACT

The present invention relates to an artificial bile duct comprising ursodeoxycholic acid (UDCA), and due to comprising UDCA, the artificial bile duct of the present invention is effective in improving differentiation into cholangiocytes and preventing formation of bile stones.

### BACKGROUND

### 1. Field of the Invention

The present invention relates to a 3D printed artificial bile duct comprising ursodeoxycholic acid (UDCA) and a manufacturing method thereof.

The present invention was derived from the following national project research service.
[Task identification number] 1711177406
[Task number] 21A0401L1
[Name of ministry] Ministry of Science and ICT (MSIT)
[Name of (specialized) institute in charge of task management] Korean Fund for Regenerative Medicine
[Research project name] Project of Korean Fund for Regenerative Medicine/Project of Korean Fund for Regenerative Medicine/Development of original technology of regenerative medicine
[Research task name] Development of method for stemness retention through identification of specific cell surface markers on next generation human hepatic stem cells
[Name of institute in charge of task performance] Hanyang University Industry-Academic Cooperation Foundation
[Research period] Jan. 1, 2023-Dec. 31, 2023

### 2. Discussion of Related Art

Treatment options for chronically progressive bile duct disorders (cholangiopathy) are limited. Liver transplantation is the main treatment for bile duct disorders, but it is not an easily accessible treatment because of the high requirements. Accordingly, research is underway to ensure that artificial bile ducts can perform biological functions in addition to the physical functions of replacing the functions of defective organs.

Meanwhile, ursodeoxycholic acid (UDCA) is a secondary bile acid that is synthesized in the liver, excreted in bile, metabolized by intestinal microorganisms, and then reabsorbed into the liver (enterohepatic recirculation). UDCA is known to play a role in improving liver function in chronic liver disease and helping the secretion of bile acids, phospholipids, and cholesterol in patients with primary biliary cirrhosis (PBC) and primary sclerosing cholangitis (PSC).

Therefore, there is an urgent need for the development of an artificial bile duct that comprises UDCA, which can prevent cell differentiation and gallstone formation in the bile duct, and is suitable for in vivo transplantation.

### 3. Prior-Art Documents

### PATENT DOCUMENTS

(Patent Document 1) KR 10-2022-0048511 A
(Patent Document 2) KR 10-2020-0075260 A
(Patent Document 3) KR 10-2018-0130625 A

### SUMMARY OF THE INVENTION

The present inventors have made extensive research efforts to develop an artificial bile duct that comprises ursodeoxycholic acid (UDCA), which can prevent cell differentiation and gallstone formation in the bile duct, and is suitable for in vivo transplantation.

As a result, the present invention was completed by developing an artificial bile duct for improvement or treatment of biliary tract diseases that comprises UDCA to allow an inner fibrous layer of the artificial bile duct to be better attached and that can help human chemically derived hepatic progenitors (hCdH) differentiate into cholangiocytes.

Accordingly, the present invention is directed to providing an artificial bile duct for improvement or treatment of biliary tract diseases that comprises an inner fibrous layer comprising a biodegradable polymer material, an outer porous foam layer comprising a biocompatible polymer material, and ursodeoxycholic acid (UDCA).

The present invention is also directed to providing a method of manufacturing an artificial bile duct.

The present invention is also directed to providing a method for improvement or treatment of biliary tract diseases that comprises transplanting an artificial bile duct into a subject that requires the same.

### BRIEF DESCRIPTION OF THE DRAWINGS

The above and other objects, features and advantages of the present invention will become more apparent to those of ordinary skill in the art by describing in detail exemplary embodiments thereof with reference to the attached drawings, in which:

FIG. 1A shows a dissolution image and graphs of pigment bile stones treated in water, 1% ethylenediaminetetraacetic acid (EDTA), 0.5% ursodeoxycholic acid (UDCA), 99% methyl tert-butyl ether (MTBE), 20 μg/ml of Paclitaxel, and 200 uM of Gemcitabine for 120 minutes at 37° C. with constant movement according to one embodiment of the present invention.

FIG. 1B shows a dissolution graph after storing cholesterol bile stones in water, 0.5% UDCA, and 100 mM of MTBE according to one embodiment of the present invention.

FIG. 1C shows a dissolution of mixed bile stones in water, 0.5% UDCA, and 100 mM of MTBE for 12 days according to one embodiment of the present invention. (Data are mean±SD (n=5) and analyzed by one-way ANOVA and Tukey test (*** p<0.001)).

FIG. 2A is a view showing a schematic diagram of a process of creating an artificial bile duct (ABD) of polyvinyl ethanol template and microinjecting polycaprolactone nanofibers comprising UDCA onto the polyvinyl ethanol template using 21 G and 27 G needles to produce an artificial bile duct using UDCA according to one embodiment of the present invention.

FIG. 2B is a graph showing results of UDCA being released of ABD for 50 days at 37° C. according to one embodiment of the present invention.

FIG. 3A is a view showing a schematic diagram of a method of differentiation of human chemically derived hepatic progenitors (hCdHs) into cholangiocytes (hCdH-Chols) according to one embodiment of the present invention.

FIG. 3B is a view comparing morphological features (scale bar is 100 μm) of hCdHs and hCdH-Chols according to one embodiment of the present invention.

FIG. 3C is a view showing scanning electron microscope (SEM) images (scale bar is 100 μm) of hCdH-Chols that are attached to an artificial bile duct comprising UDCA according to one embodiment of the present invention.

FIG. 3D is a view showing gene expression levels of markers specific to hCdH-Chols according to one embodiment of the present invention (Data are mean±SD (n=5) and analyzed by one-way ANOVA and Tukey test (*** p<0.001)).

FIG. 3E is a view showing results of immunofluorescence staining of biliary cell (AQP1, KRT19, CFTR, KRT7, and SOX9) markers in hCdH-Chols (scale bar is 100 μm) according to one embodiment of the present invention.

FIG. 3F is a view showing results of functional tests of bile acid analog 5 (6)-carboxy-2′,7′-dichlorofluorescein (CDF) (scale bar is 100 μm) according to one embodiment of the present invention.

FIG. 4A is a view showing an animal model for transplantation of a UDCA-comprising ABD according to one embodiment of the present invention.

FIG. 4B is a graph showing a comparison of survival rates after ABD transplantation according to one embodiment of the present invention.

FIG. 4C is a view showing a state after ABD transplantation surgery according to one embodiment of the present invention.

FIG. 4D shows graphs confirming liver enzymes according to one embodiment of the present invention.

FIG. 4E are graphs showing results of measuring bile acid, bilirubin, and cholesterol levels in bile according to one embodiment of the present invention.

### DETAILED DESCRIPTION OF EXEMPLARY EMBODIMENTS

Hereinafter, the present invention will be described with reference to examples and comparative examples in detail. However, the present invention is not limited to these examples.

Hereinafter, the present invention will be described in more detail.

One aspect of the present invention is an artificial bile duct for improvement or treatment of biliary tract diseases that comprises an inner fibrous layer comprising a biodegradable polymer material, an outer porous foam layer comprising a biocompatible polymer material, and ursodeoxycholic acid (UDCA).

In the present invention, the biodegradable polymer material must be capable of self-degradation after in vivo transplantation of the ABD. In one embodiment, the inner fibrous layer is made of a biodegradable polymer material for, after the in vivo transplantation of the ABD, the fibrous layer to degrade and disappear on its own after cells grow and the tissue is restored. The biodegradable polymer material may be one or more selected from the group consisting of polycaprolactone (PCL), polylactic acid (PLA), polyglycolic Acid (PGA), poly(lactic-co-glycolic acid), polyphosphates, polyphosphazene, polyphosphonate, poly(sebacic acid), polydianones, poly (E-caprolactone), polyhydroxybutyrate, poly B-maleic acid, polyaminoacid, polycynoacrylate, polyurethanes, polyorthoesters, and polycarprolaction-co-lactide, and for example, the biodegradable polymer material may be PCL but is not limited thereto.

In the present invention, the outer porous foam layer should be able to continuously perform a physical function after the in vivo transplantation of the ABD and thus should be made of a biocompatible material. In one embodiment, since the ABD disclosed herein is for in vivo transplantation, the porous foam layer is made of a biocompatible polymer material. The biocompatible polymer material may be one or more selected from the group consisting of polyethylene, polypropylene, polytetrafluoroethylene, poly(ethylene terephthalate), poly(methyl methacrylate), poly(2-hydroxyethyl methacrylate), poly(N-isopropylacrylamide), polysiloxane, and polyurethanes, and for example, the biocompatible polymer material may be polyurethane but is not limited thereto.

In the present specification, “ursodeoxycholic acid (UDCA)” is a secondary bile acid that is synthesized in the liver, excreted in bile, metabolized by intestinal microorganisms, and then reabsorbed into the liver (enterohepatic recirculation).

In the present invention, “ursodeoxycholic acid (UDCA)” may have the structure of Chemical Formula 1.

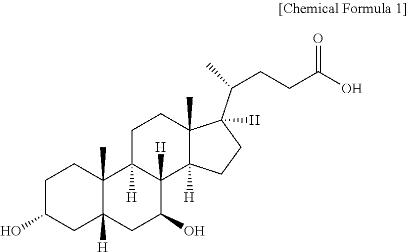

In an example below, when bile stones were treated with UDCA, MTBE, paclitaxel, and gemcitabine and then bile stone dissolution capacities were evaluated, it was confirmed that the stones treated with UDCA dissolved the most.

In the present invention, the ABD may further comprise a fusion layer in which the inner fibrous layer and the outer porous foam layer are fused, but the present invention is not limited thereto.

The fusion layer may be formed by a remaining solvent dissolving nanofibers of the inner fibrous layer when the inner fibrous layer is immersed in a salt-suspended biocompatible polymer material, but the present invention is not limited thereto.

In the present invention, the inner fibrous layer may be electrospun using needles with a diameter of 15 G or more, and for example, may be electrospun using needles with a diameter ranging from 15 G to 40 G, but the present invention is not limited thereto.

In addition, in an example below, ABD were manufactured by controlling a fiber diameter of the inner fibrous layer by electrospinning using 21 G and 27 G needles. The ABD comprising fibers electrospun using 21 G and 27 G needles all showed a continuous release of UDCA for 50 days. Electrospinning using needles with a diameter of 15 G or more, for example, a diameter ranging from 15 G to 40 G, was performed to achieve a fiber diameter most suitable for releasing UDCA.

In the present invention, the inner fibrous layer may be characterized as comprising hepatic progenitors reprogrammed by a medium composition that comprises a hepatocyte growth factor (HGF), A83-01, and CHIR99021 and is for reprogramming human adult hepatocytes to hepatic progenitors, but the present invention is not limited thereto.

In an example below, whether differentiation of human chemically derived hepatic progenitors (hCdHs) into cholangiocytes was promoted was evaluated. The differentiation of hCdHs into cholangiocytes was promoted, and even after a period of differentiation, the cholangiocytes (hCdH-Chols) expressed high levels of cholangiocyte markers.

In addition, in an example below, the last group of rabbits into which the ABD comprising UDCA and hCdH-Chols was transplanted showed the highest survival rate. Further, it can be confirmed that the ABD is effective in preventing bile stone formation and elevation of liver enzymes.

In the present invention, the biliary tract diseases may be one or more selected from the group consisting of primary biliary cirrhosis (PBC), primary sclerosing cholangitis (PSC), cystic fibrosis, biliary atresia, and cholangiocarcinoma, but are not limited thereto.

Another aspect of the present invention is a method of manufacturing an ABD that comprises the following steps:
(a) manufacturing a patient-customized 3D template or a 3D template of any shape using a water-soluble polymer material;
(b) manufacturing a fiber-deposited 3D template by mixing ursodeoxycholic acid (UDCA) with a biodegradable polymer material and performing electrospinning on the 3D template;
(c) forming a porous foam layer on an outer side of the 3D template by dip-coating the fiber-deposited 3D template made in step (b) in a salt-suspended biocompatible polymer material;
(d) fabricating a tubular structure by immersing the material formed in step (c) in water and removing salt particles and the 3D template;
(e) seeding cells on a fiber-deposited inner fibrous layer.

In the present invention, the water-soluble polymer material may be one or more selected from the group consisting of a polyvinyl alcohol (PVA) filament and polyethylene glycol (PEG) but is not limited thereto.

In the present invention, the biodegradable polymer material may be one or more selected from the group consisting of polycaprolactone (PCL), polylactic acid (PLA), polyglycolic acid (PGA), poly(lactic-co-glycolic acid), polyphosphates, polyphosphazene, polyphosphonate, poly(sebacic acid), polydianones, poly (E-caprolactone), polyhydroxybutyrate, poly B-maleic acid, polyaminoacid, polycynoacrylate, polyurethanes, polyorthoesters, and polycarprolaction-co-lactide, but is not limited thereto.

In the present invention, the biocompatible polymer material may be one or more selected from the group consisting of polyethylene, polypropylene, polytetrafluoroethylene, poly(ethylene terephthalate), poly(methyl methacrylate), poly(2-hydroxyethyl methacrylate), poly(N-isopropylacrylamide), polysiloxane, and polyurethanes, but is not limited thereto.

Still another aspect of the present invention is a method for improvement or treatment of biliary tract diseases that comprises transplanting an artificial bile duct into a subject that requires the same.

Advantages and features of the present invention and methods of achieving the same should become clear from embodiments described in detail below. However, the present invention is not limited to the embodiments disclosed below and may be implemented in various different forms. The present embodiments are only provided to make the disclosure of the present invention complete and completely inform those of ordinary skill in the art to which the present invention pertains of the scope of the invention, and the scope of the present invention is defined only by the scope of the claims.

Hereinafter, the present invention will be described in detail using examples. However, the following examples only illustrate the present invention, and the content of the present invention is not limited to the following examples.

### EXAMPLES

### Terms

For a clear understanding of the present invention, terms are defined as follows.

Hepatic progenitor cells chemically derived from human hepatocytes: Human chemically-derived hepatic progenitors (hCdHs)

Human liver cells: human primary hepatocytes, hPHs

Biliary epithelial cells: Cholangiocytes

Hepatic progenitor-derived cholangiocytes: Human chemically-derived hepatic progenitors-cholangiocytes, hCdH-Chols

Percentage (%) concentration may be expressed as % (w/w), % (w/v), % (v/v), and % (v/w).

### Manufacture Example 1. Manufacture of Artificial Bile Duct

Manufacture of the artificial bile duct disclosed herein was carried out by steps of 3D printing of a template, fiber coating by electrospinnning, dip coating in a polymer solution, and removal of the template and particles as described in a recent publication (transplantation of patient-specific bile duct bioengineered with chemically reprogrammed and microtopographically differentiated cells, Bioeng Transl Med. 2021 Sep. 3; 7 (1): e10252. doi: 10.1002/btm2.10252. eCollection 2022 January).

In manufacturing a 3D template, computer-aided design (CAD) based on magnetic resonance imaging (MRI) data was used. Structures of the biliary tree were three-dimensionally reconstructed from 2D magnetic resonance cholangiopancreatography (MRCP) images and post-processed in a 3D printable file format (stereolithrography (STL)). As the 3D template, a polyvinyl alcohol (PVA) filament was printed using a 3D printer driven by a material compression method. A G-code for printing was generated using the open-source software Cura. Parameters of a nozzle inner diameter, a nozzle temperature, and a layer thickness were set to 0.25 mm, 170° C., and 0.15 mm, respectively. To exclude surface roughness of the printed template, the template was ultrasonicated by immersing it in warm distilled water at 50° C. for 1 minute.

For the next step, electrospinning was performed by mixing a polycaprolactone (PCL) (MW 80,000; Sigma-Aldrich) solution and 0.5% ursodeoxycholic acid (UDCA) while rotating the 3D printed PVA template. PCL granules were dissolved in a mixture of methylene chloride and dimethylformaldehyde (DMF) at a ratio of 3:1. The solution concentration was set to 18% (w/v) to generate nano-scale and micro-nano-scale hybrid electrospun mats. Next, the fiber-deposited template was immersed in a salt-suspended polyurethane solution for dip-coating, and then salt particles were leached to form a porous foam layer around the fiber template. For the dip-coating step, the fiber-coated template was immersed in a salt-suspended thermoplastic polyurethane (TPU) (Daerim Chemical) solution. TPU granules were dissolved in DMF at a concentration of 15% (w/v). Then, salt particles sieved through a 45-micrometer mesh were mixed with the TPU solution at a concentration of 400% (w/w). The coated portion was dried and then dipped in ultrasonicated water for 2 minutes to filter out the salt particles. First, for the ‘Cys-(8Asp)-(16Arg)-Cys’ peptide, a solid-phase Fmoc peptide synthesis method, which is a synthesis method in which each amino acid is elongated one by one according to a set sequence order, may be used, and amino acids in which the α-amino group is protected with an Fmoc (Fluorenylmethoxycarbonyl, 9-fluorenylmethoxycarbonyl) group are used. After the peptide chain elongation is complete, a free peptide is obtained by treatment with trifluoroacetic acid (TFA).

The final artificial bile duct with a fiber form on a surface of an innermost layer was obtained after dissolving the 3D template in distilled water and removing it. A 3D artificial bile duct was obtained by removing the PVA template by dipping it in ultrasonicated water at 50° C. for 30 minutes. During the PVA template removal process, a tip of the coated template was slightly cut to create an inlet for water penetration.

### Manufacture Example 2. Differentiation of Cholangiocytes (hCdH-Chols) Derived from Human Chemically Derived Hepatic Progenitors (hCdHs)

In manufacturing a 3D artificial bile duct disclosed in the present specification, cholangiocytes (hCdH-Chols) derived from human chemically derived hepatic progenitors (hCdHs) were used. Because primary cholangiocytes from humans and other mammals are difficult to isolate and propagate in vitro, human chemically derived hepatic progenitors (hCdHs) were utilized for biliary epithelium modeling. To generate hCdHs, human adult hepatocytes isolated from healthy donor livers were directly reprogrammed into bi-potent progenitors through combinatorial treatment with two small molecules, A83-01 and CHIR99021, in the presence of a hepatocyte growth factor (HGF) as described in a recent publication (Y. Kim, K. Kang, S. B. Lee, D. Seo, S. Yoon, S. J. Kim, K. Jang, Y. K. Jung, K. G. Lee, V. M. Factor, J. Jeong, D. Choi, J Hepatol 2019, 70, 97.). A cholangiocyte differentiation protocol was applied when hCdHs reached a passage 3. The hCdHs cultured for 14 days in a cholangiocyte differentiation medium comprising Na-taurocholate (100 uM) and CHIR99021 (10 mM) in the presence of a HGF for hCdH-Chols differentiation.

### Example 1. Evaluation of Bile Stones Dissolution Capacity

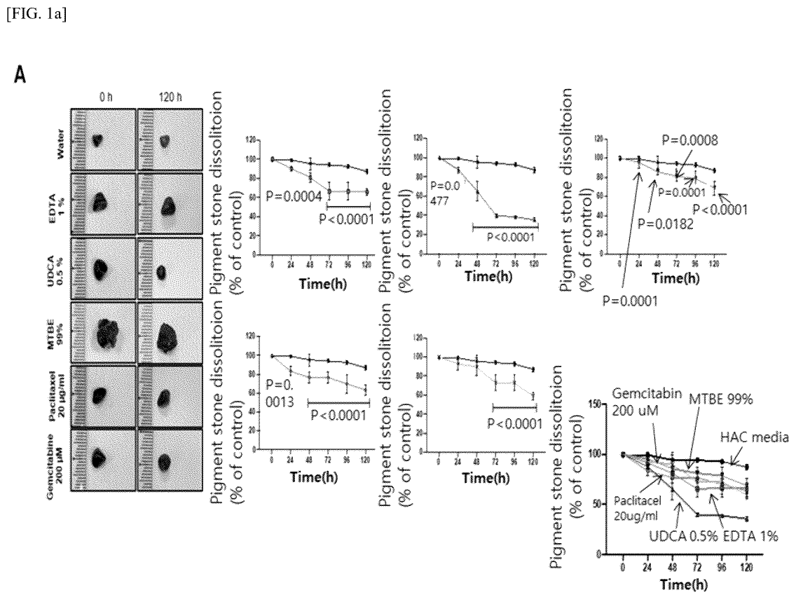

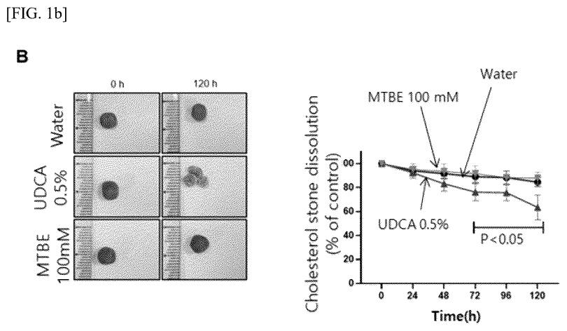

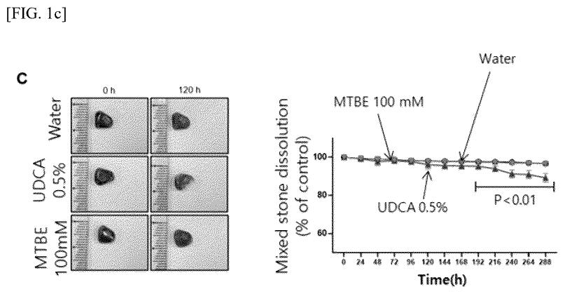

Pigment bile stones were stored in water, 1% ethylenediaminetetraacetic acid (EDTA), 0.5% ursodeoxycholic acid (UDCA), 99% methyl tert-butyl ether (MTBE), 20 μg/ml of Paclitaxel, and 200 uM of Gemcitabine, observed for 120 hours at 37° C. with constant movement, and the weights of the bile stones were measured every 24 hours to evaluate bile stone dissolution capacity (FIG. 1).

As a result of the experiment, the dissolution capacity of the bile stones in UDCA was significantly increased compared to other solvents.

### Example 2. Evaluation of Drug Delivery According to Diameter of Inner Fibrous Layer

In manufacture using the methods of the manufacture examples described above, ABDs were manufactured by controlling a fiber diameter of the inner fibrous layer by electrospinning using 21 G and 27 G needles. In addition, a release rate of UDCA from the inner fibrous layer of the artificial bile duct was measured and evaluated at 37° C. for 50 days.

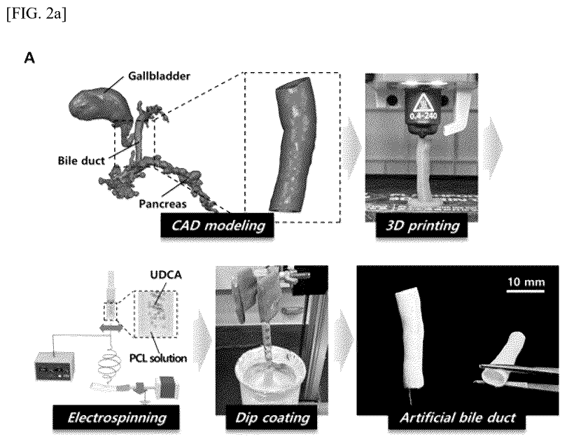

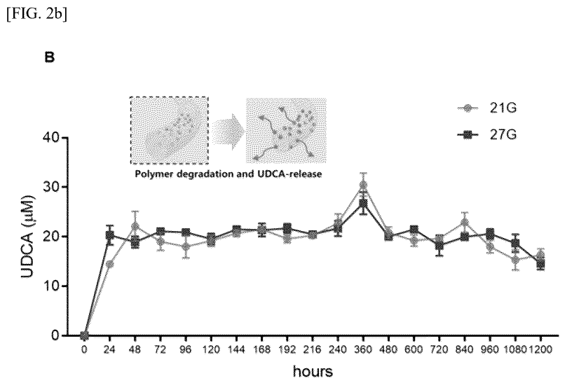

As a result of the experiment, the artificial bile ducts comprising the inner fibrous layer electrospun using 21 G and 27 G needles all showed a continuous release of UDCA for at least 50 days (1,200 hours) (FIG. 2).

### Example 3. Evaluation of Whether Differentiation into Cholangiocytes is Promoted by UDCA

The ABDs comprising the inner fibrous layer electrospun using 21 G and 27 G needles by the method of Example 2 were treated with UDCA, and after 50 days, whether differentiation into cholangiocytes using human chemically derived hepatic progenitors (hCdHs) is promoted was evaluated. As a control group, a 3D ABD untreated with UDCA was compared.

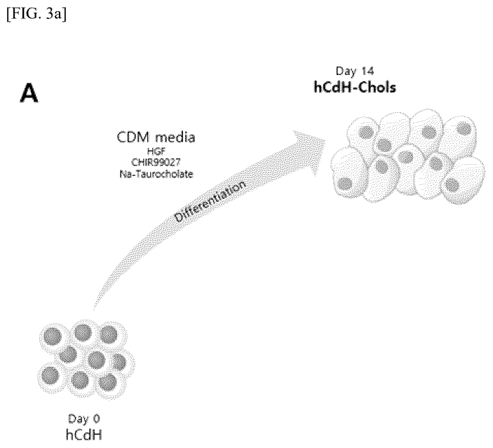

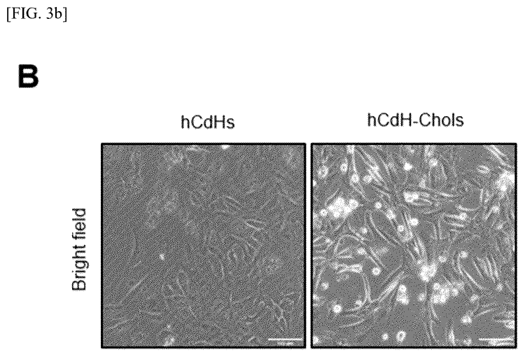

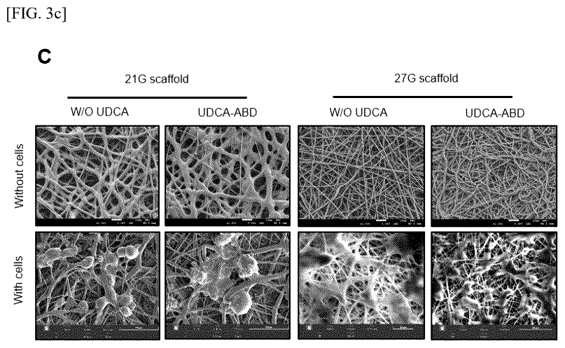

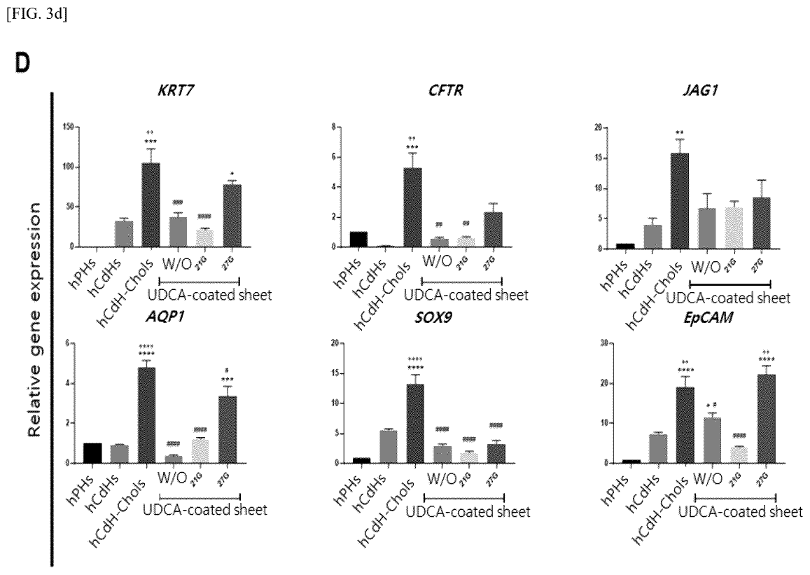

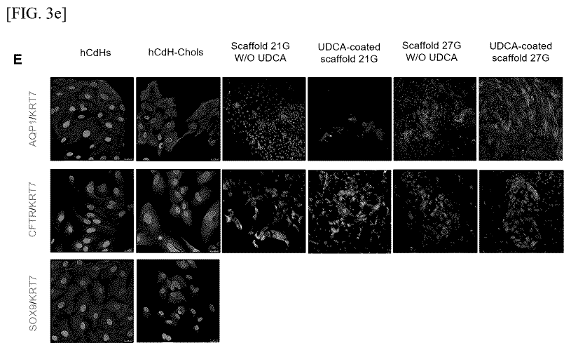

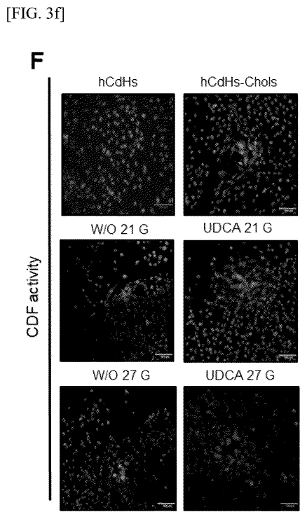

As a result of the experiment, when UDCA was present in the inner fibrous layer of the ABD, nanofibers of the inner fibrous layer better attached cells to the bile ducts. In addition, UDCA promoted differentiation of hepatic progenitors (hCdHs) into cholangiocytes (hCdH-Chols), and even after a period of differentiation, the cholangiocytes (hCdH-Chols) expressed high levels of cholangiocyte markers (FIG. 3).

### Example 4. Animal Model Evaluation

The manufactured 3D ABD comprising UDCA was transplanted into typical rabbit models with biliary tract defects to evaluate the efficacy of hCdH-Chols. For biliary tract defects, a 5-mm-long piece was removed from the healthy bile duct of a rabbit, and an ABD construct filled with a 10-mm piece of hCdH-Chols was transplanted.

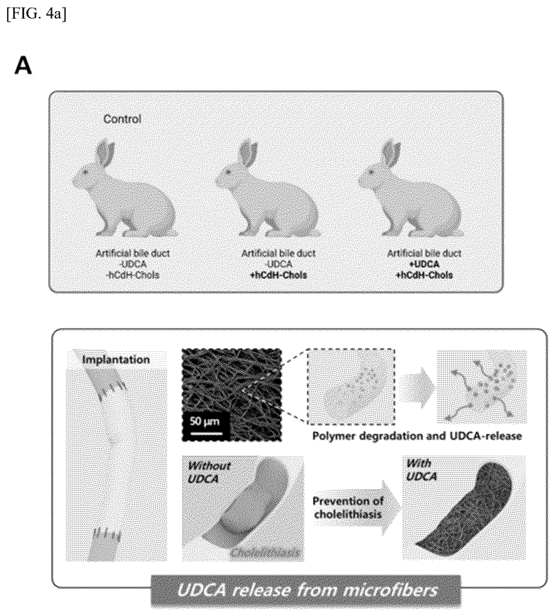

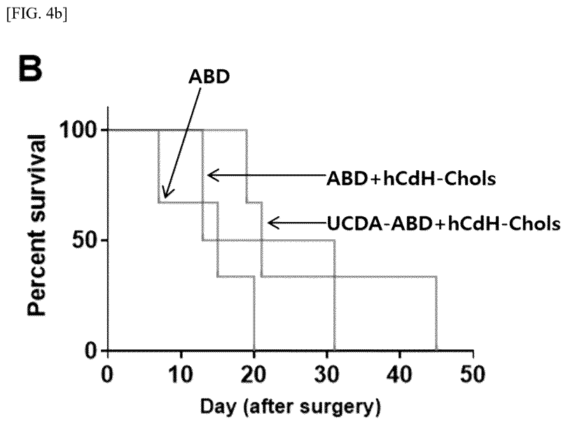

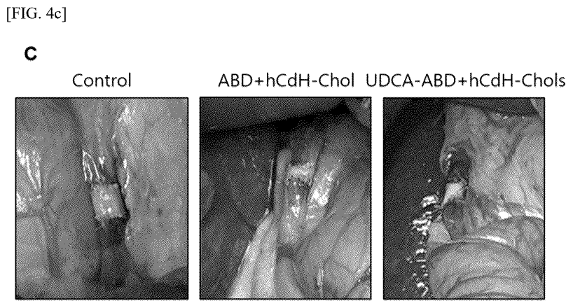

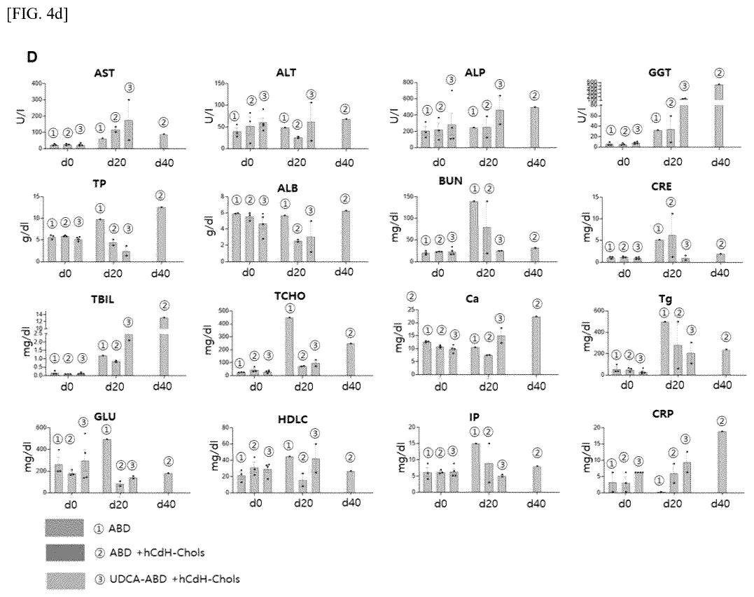

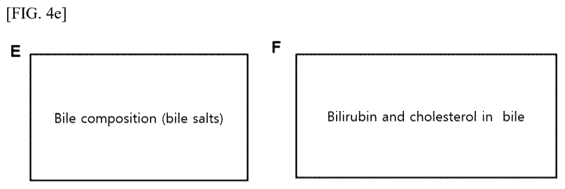

As a result of the experiment, due to differentiation into cholangiocytes (hCdH-Chols), a group of rabbits into which the ABD comprising hCdH-Chols was transplanted showed a higher survival rate than a group of rabbits into which only an ABD was transplanted (FIG. 4). In addition, liver function was also shown to improve in the hCdH-Chols group over time. The ABD of hCdH-Chols comprising UDCA was found to be effective in preventing the formation of bile stones.

While the invention has been shown and described with reference to certain exemplary embodiments thereof, it will be understood by those skilled in the art that various changes in form and details may be made therein without departing from the spirit and scope of the invention as defined by the appended claims.

What is claimed is:

1. An artificial bile duct for improvement or treatment of biliary tract diseases, the artificial bile duct comprising:
an inner fibrous layer comprising a biodegradable polymer material;
an outer porous foam layer comprising a biocompatible polymer material; and
ursodeoxycholic acid (UDCA).

2. The ABD of claim 1, wherein the biodegradable polymer material having a fibrous form is one or more selected from the group consisting of polycaprolactone (PCL), polylactic acid (PLA), polyglycolic Acid (PGA), poly(lactic-co-glycolic acid), polyphosphates, polyphosphazene, polyphosphonate, poly(sebacic acid), polydianones, poly (E-caprolactone), polyhydroxybutyrate, poly B-maleic acid, polyaminoacid, polycynoacrylate, polyurethanes, polyorthoesters, and polycarprolaction-co-lactide.

3. The ABD of claim 1, wherein the biocompatible polymer material is one or more selected from the group consisting of polyethylene, polypropylene, polytetrafluoroethylene, poly(ethylene terephthalate), poly(methyl methacrylate), poly(2-hydroxyethyl methacrylate), poly(N-isopropylacrylamide), polysiloxane, and polyurethanes.

4. The ABD of claim 1, further comprising a fusion layer in which the inner fibrous layer and the outer porous foam layer are fused.

5. The ABD of claim 1, wherein the inner fibrous layer is electrospun using needles with a diameter of 15 G or more.

6. The ABD of claim 1, wherein the inner fibrous layer comprises hepatic progenitors reprogrammed by a medium composition that comprises a hepatocyte growth factor (HGF), A83-01, and CHIR99021 and is for reprogramming human adult hepatocytes to hepatic progenitors.

7. The artificial bile duct of claim 1, wherein the biliary tract diseases are one or more selected from the group consisting of primary biliary cirrhosis (PBC), primary sclerosing cholangitis (PSC), cystic fibrosis, biliary atresia, and cholangiocarcinoma.

8. A method of manufacturing the ABD of claim 1, the method comprising:
(a) manufacturing a patient-customized 3D template or a 3D template of any shape using a water-soluble polymer material;
(b) manufacturing a fiber-deposited 3D template by mixing ursodeoxycholic acid (UDCA) with a biodegradable polymer material and performing electrospinning on the 3D template;
(c) forming a porous foam layer on an outer side of the 3D template by dip-coating the fiber-deposited 3D template made in step (b) in a salt-suspended biocompatible polymer material;
(d) fabricating a tubular structure by immersing the material formed in step (c) in water and removing salt particles and the 3D template; and
(e) seeding cells on a fiber-deposited inner fibrous layer.

9. A method for improvement or treatment of biliary tract diseases, the method comprising transplanting the artificial bile duct of claim 1 into a subject that requires the same.
